(19) United States
(12) Patent Application Publication
(10) Pub. No.: US 2005/0234499 A1
(43) Pub. Date: Oct. 20, 2005

(54) Multi-lumen balloon catheter including manifold

(75) Inventors: Gregory Olson, Elk River, MN (US); August Powell, Zimmerman, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: SCIMED LIFE SYSTEMS, INC.

(21) Appl. No.: 10/827,583

(22) Filed: Apr. 19, 2004

(51) Int. Cl. A61M 29/00

(52) U.S. Cl. 606/192

(57) ABSTRACT

Alternative designs, materials, and methods of making and using catheter manifolds. Some embodiments are related to a balloon catheter including a catheter shaft having a proximal portion and a distal portion. The shaft includes an outer tubular member defining a lumen and having a proximal end and a distal end. The shaft also includes an inner tubular member defining a lumen and having a proximal end and a distal end. The inner tubular member is disposed at least partially within the outer tubular member such that the proximal end of the inner tubular member extends proximally from the proximal end of the outer tubular member. A catheter manifold is molded about a portion of the outer tubular member and a portion of the inner tubular member, and a balloon assembly attached to the distal portion of the shaft such that the balloon assembly is in fluid communication with at least one of the lumens.

### FIELD

The invention is related to the field of medical devices, and more particularly, to catheters including medical balloon catheters including a manifold.

### BACKGROUND

It is generally known to provide a manifold on a medical catheter such as a balloon catheter. However, providing a manifold on a catheter can add significant expense and difficulty to catheter fabrication. A number of different catheter manifold structures and assemblies, and methods of making and using catheter manifolds are known, each having certain advantages and disadvantages. However, there is an ongoing need to provide alternative structures, assemblies, and methods for making and using catheter manifolds.

### SUMMARY OF SOME EMBODIMENTS

In some aspects, the invention relates to alternative designs, materials, and methods of making and using catheter manifolds.

For example, in some embodiments, the invention relates to a balloon catheter including a catheter shaft having a proximal portion and a distal portion. The shaft includes an outer tubular member defining a lumen and having a proximal end and a distal end. The shaft also includes an inner tubular member defining a lumen and having a proximal end and a distal end. The inner tubular member is disposed at least partially within the outer tubular member such that the proximal end of the inner tubular member extends proximally from the proximal end of the outer tubular member. A catheter manifold is molded about a portion of the outer tubular member and a portion of the inner tubular member, and a balloon assembly is attached to the distal portion of the shaft such that the balloon assembly is in fluid communication with at least one of the lumens.

Some example embodiments relate to a method of making a balloon catheter. The method includes providing a multi-lumen catheter shaft, for example as discussed above, wherein the inner tubular member is disposed at least partially within the lumen of the outer tubular member such that the proximal end of the inner tubular member extends proximally from the proximal end of the outer tubular member. The method also includes molding a manifold about the proximal portion of the multi-lumen catheter shaft such that the manifold is disposed about both the proximal end of the inner tubular member and the proximal end of the outer tubular member. Additionally, a balloon assembly is attached to the distal portion of the shaft such that the balloon assembly is in fluid communication with at least one of the lumens.

The above summary of some embodiments is not intended to describe each disclosed embodiment or every implementation of the present invention. The Figures, and Detailed Description which follow more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a partial side view of one example embodiment of a balloon catheter including an example manifold;

FIG. 2 is a partial cross sectional view of the balloon catheter of FIG. 1;

FIG. 3 is a cross sectional side view of the manifold of the catheter of FIGS. 1 and 2;

FIG. 4A is a schematic side view of an example mold that may be used in making a manifold;

FIG. 4B is a top view of a portion of a mold showing the proximal end of the inner and outer tubular members of the catheter shaft and core pins disposed within the mold cavity prior to the introduction of molding material;

FIG. 5 is a cross sectional view taken along line 5-5 in FIG. 4B;

FIG. 6 is a cross sectional view taken along line 6-6 in FIG. 4B;

FIG. 7 is a top view of a portion of the mold of FIG. 4B showing the molding material introduced into the mold cavity;

FIG. 8 is a cross sectional side view of another example embodiment of a manifold for a catheter similar to that shown in the figures above, but including an additional layer disposed at least partially between the catheter shaft and the manifold; and

FIG. 9 is a cross sectional view taken along line 9-9 in FIG. 8.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF SOME EMBODIMENTS

For the following defined terms, the definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). The term “about” may include numbers that are rounded to the nearest significant figure.

Weight percent, percent by weight, wt %, wt-%, % by weight, and the like are synonyms that refer to the concentration of a substance as the weight of that substance divided by the weight of the composition and multiplied by 100.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used herein the term “overmolding” refers to a process wherein a molding material is molded over and/or about at least a portion of an existing item. Overmolding includes processes where some of the molding material may pass into or between existing elements as well.

The following detailed description of some embodiments should be read with reference to the drawings, wherein like reference numerals indicate like elements throughout the several views. The drawings, which are not necessarily to scale, depict some example embodiments and are not intended to limit the scope of the invention. Those skilled in the art and others will recognize that many of the examples provided have suitable alternatives which may also be utilized.

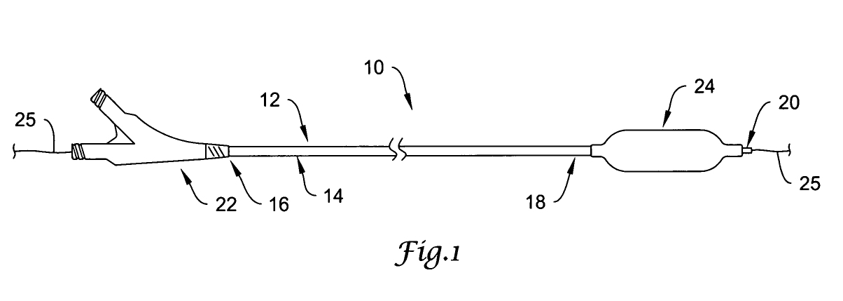

Refer now to FIG. 1, which is a partial side view of one example embodiment of a catheter 10 including a manifold 22. In the embodiment shown, the catheter 10 is a balloon catheter, for example; an over-the-wire (OTW) balloon catheter that may be configured for advancement over a guidewire 25, or other such device. The catheter 10 can include a shaft assembly 12 including a proximal portion 14 having a proximal end 16, and a distal portion 18 having a distal end 20. A manifold 22 can be disposed adjacent the proximal end 16 of the shaft assembly 12, and one or more deployable balloon assembly 24 can be disposed adjacent the distal end 20 of the shaft assembly 12.

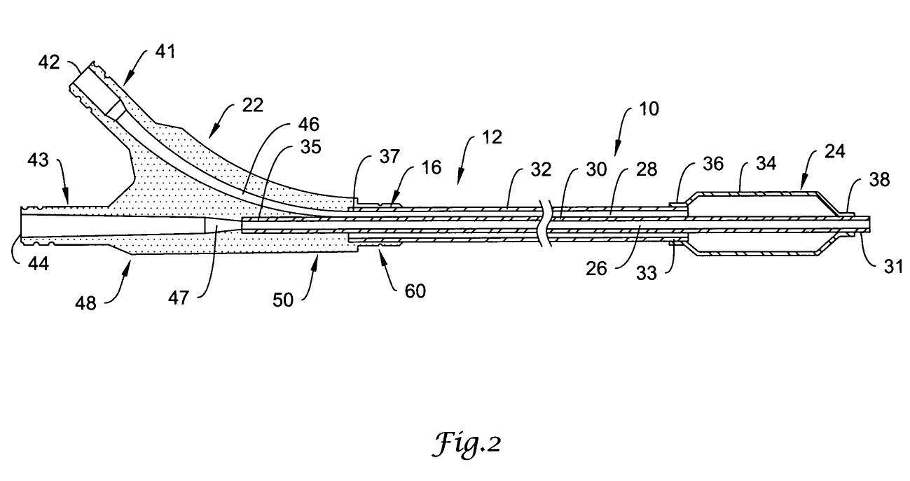

Referring now to FIG. 2, the shaft assembly 12 can include at least two or more lumens extending therein. The embodiment shown includes two lumens, for example a device lumen 26 and an inflation lumen 28. The device lumen 26 extends the length of the shaft assembly 12, and may be adapted to receive a device 25, such as a guide wire, or other such medical device, as is generally known in the art. In general, the device lumen 26 can be accessed through the manifold 22 such that the device 25 can extend through the manifold 22 into the lumen 26. The inflation lumen 28 allows fluid communication between the manifold 22 and the deployable balloon assembly 24. In general, the proximal end of the inflation lumen 28 can be put into fluid communication with an inflation source via the manifold 22, and the distal end of the inflation lumen 28 is in fluid communication with the interior of the deployable balloon assembly 24.

The shaft assembly 12 can include a generally coaxial design including a plurality of tubular members generally coaxially disposed to form the lumens 26/28. In the generally co-axial design shown, the shaft assembly 12 can include an inner tubular member 30 and an outer tubular member 32. The inner tubular member 30 defines the device lumen 26, the outer tubular member 32 is disposed about the inner tubular member 32 to define the annular inflation lumen 28 between the inner and outer tubular members 30/32. In at least some embodiments, the inner and outer tubular members 30/32 are two separate and distinct structures, and the inner tubular member 30 is disposed within and may be attached to the outer tubular member 32 to create the shaft assembly 12. In the embodiment shown, the proximal end 35 of the inner tubular member 30 extends proximally beyond the proximal end 37 of the outer tubular member 32, and the manifold 22 is disposed about the proximal portion 14 of the shaft assembly 12. As such, the manifold 22 can be disposed about and/or attached directly to a portion of the inner tubular member 30 adjacent the proximal end 35 thereof, and disposed about and/or attached directly to a portion of the outer tubular member 32 adjacent the proximal end 37 thereof. Such an arrangement can provide for good attachment of the manifold 22 to both the inner and outer tubular members 30/32 of the shaft 12. This can provide a mechanism for maintaining the longitudinal positioning of the inner and outer tubular members 30/32 relative to one another. In at least some embodiments, the proximal end 35 of the inner tubular member 30 extends proximally beyond the proximal end 37 of the outer tubular member 32 by a length in the range of about 0.5 cm or more, or by a length in the range of about 0.5 to about 3 cm.

Also, in some embodiments, the distal end 31 of the inner tubular member 30 may extend distally beyond the distal end 33 of the outer tubular member 32 such that when the balloon assembly 24 is attached, as discussed below, the inflation lumen 28 is in fluid communication with the interior of the balloon assembly 24. However, it is contemplated that in other alternative embodiments, the distal end 31 of the inner tubular member 30 may not extend distally beyond the distal end 33 of the outer tubular member 32. For example, it is contemplated that in some arrangements, the distal ends 31/33 may end at generally the same point, or the distal end 33 may extend distally beyond the distal end 31, depending at least somewhat upon the desired distal construction and/or balloon assembly used in the catheter 10.

The shaft assembly 12, including the inner and outer tubular members 30/32, may have conventional sizes, shapes, dimensions, and may be made of conventional materials using known shaft construction methods and/or techniques for making catheters, such as balloon catheters. In some embodiments, the tubular members 30/32 may be single or multi-layered shafts, and may include reinforcing braids, markers, such as radiopaque markers, or other such structures, many of which are generally known. The portions of the tubular members 30/32 to which the manifold is to become bonded and/or overmolded may be chosen of suitable materials or include structure for such bonding and/or overmolding. For example, in some embodiments, the tubular members 30/32 may comprise one or more polymers, metals, and or composite material. Some examples or materials include polyoxymethylene (POM), polybutylene terephthalate (PBT), polyether block ester, polyether block amide (PEBA), fluorinated ethylene propylene (FEP), polyethylene (PE), polypropylene (PP), polyvinylchloride (PVC), polyurethane, polytetrafluoroethylene (PTFE), polyether-ether ketone (PEEK), polyimide, polyamide, polyphenylene sulfide (PPS), polyphenylene oxide (PPO), polysufone, nylon, perfluoro(propyl vinyl ether) (PFA), polyether-ester, polymer/metal composites, etc., or mixtures, blends or combinations thereof, or the like. One example of a suitable polyether block ester is available under the trade name ARNITEL, and one suitable example of a polyether block amide (PEBA) is available under the trade name PEBAX®, from ATOMCHEM POLYMERS, Birdsboro, Pa. One example of a suitable polyoxymethylene (POM) is Delrin™ commercially available from Dow Chemicals. In at least some embodiments, the shaft 12 is suitable for intravascular navigation as in, for example, conventional diagnosis and/or treatment of blood clots, strokes, aneurisms, angiography, angioplasty, endoscopic procedures, stent deployment procedures, and/or the like.

In this regard, the inner and outer tubular members 30/32 may be sized as desired, for the particular application and/or applications for which the catheter 10 is designed. For example, the outer tubular member 32 may be sized such that it may be navigated to the desired location within the anatomy. The inner tubular member 30 can be sized such that it can be disposed within the outer tubular member 32. As such, the device lumen 26, defined by the inner tubular member 30, can be generally smaller in cross section than the lumen defined by the outer tubular member 32. In some example embodiments, the inner tubular member 30 may have an inner diameter in the range of about 0.02 to about 0.2 cm, and a wall thickness in the range of about 0.005 to about 0.03 cm. In some example embodiments, the outer tubular member 32 may have a have an inner diameter in the range of about 0.05 to about 0.6 cm, and a wall thickness in the range of about 0.005 to about 0.03 cm. It should be understood that these sizes, as with all other specific sizes given herein, are provided by way of example only, and that other sizes may be used in other embodiments.

Additionally, the shaft 12 may have a length, as desired, such that the shaft 12 is useful for the particular application and/or applications for which the catheter 10 is designed. In some example embodiments, the shaft assembly 12 may have a length in the range of about 10 to about 300 cm.

The lengths of the inner and outer tubular members 30/32 can be such that when the inner tubular member 30 is disposed at least partially within the outer tubular member 32 in a desired configuration, the proximal end 35 of inner tubular member 30 can be disposed such that it extends proximally beyond the proximal end 37 of the outer tubular member 32, as discussed above. The actual lengths of the inner and outer tubular members 30/32, are at least somewhat dependent upon the desired arrangement of the inner and outer tubular members 30/32 over the remainder of the shaft 12, for example, near the distal end of the shaft assembly 12. In certain embodiments, the desired arrangement may require that the distal end 31 of the inner tubular member 30 extends distally from the distal end 33 of the outer tubular member 32. In such embodiments, it is generally true that the inner tubular member 30 will be longer than the outer tubular member 32. In other embodiments, however, it is contemplated that the desired arrangement may not require that the distal end 31 of the inner tubular member 30 extends distally from the distal end 33 of the outer tubular member 32. Therefore, in such embodiments, the inner tubular member 30 may not be longer, but in fact may be the same length, or may be shorter than the outer tubular member 32. In some example embodiments, the inner tubular member 30 may have a length in the range of about 10 to about 300 cm, and the outer tubular member 32 may have a length in the range of about 10 to about 300 cm.

Also, while the tubular members 30/32 illustrated in the Figures have generally circular cross sections, this is not necessary in all embodiments. In other embodiments, the tubular members 30/32 may have other cross-sectional shapes, for example, oval, square, rectangular, or other polygon shapes, or the like. Additionally, while the embodiments shown include two tubular members 30/32 defining two lumens 26/28, it should be understood that in other embodiments, the shaft 12 may include one or more additional tubular members which may define one or more additional lumens within the shaft 12. Such additional tubular members may, for example, be disposed in a generally coaxially arrangement or in a side by side arrangement with the inner tubular member 30 within the outer tubular member 32.

The balloon assembly 24 disposed at the distal portion of the shaft 12 may also include conventional materials and/or dimensions, and known methods and/or techniques of making or attaching the balloon assembly 24 may be used. In the embodiment shown, the deployable balloon assembly 24 may include an expandable balloon portion 34, a proximal balloon waist 36, and a distal balloon waist 38. At the proximal balloon waist 36, the balloon portion 34 is connected to the outer surface of the outer tubular member 32 adjacent the distal end 33 of the outer tubular member 32. Any of a broad variety of suitable attachment means may be used, for example, an adhesive, a thermal bond, a mechanical bond, such as mechanically interlocking structures, friction fit, or the like. The inner tubular member 30 extends distally beyond the distal end 33 of the outer tubular member 32, through the interior of the balloon portion 34, and to a point distal of the expandable balloon portion 34. At the distal balloon waist 38, the expandable balloon portion 34 is connected to the outer surface of the inner tubular member 30 adjacent the distal end 31 of the inner tubular member 30. Again, any of a broad variety of suitable attachment means may be used, for example, those discussed above. It should be understood that the embodiment shown is a schematic representation of one example embodiment, and that a broad variety of alternative structures and arrangements can be used to create the shaft assembly 12 including a deployable balloon assembly 24. Some examples of shaft assembly constructions are disclosed in U.S. Pat. No. 5,047,045 to Arney et al., which is incorporated herein by reference.

The manifold 22 can be of unitary or monolithic construction, meaning, for example, that it is a single or unitary piece of material. The manifold 22 can be overmolded or otherwise formed or created directly onto a part of the proximal portion 14 of the shaft 12 adjacent and/or about the proximal end 16 of the shaft 12. Due to the staggered arrangement of the inner and outer tubular members 30/32 adjacent the proximal end 16 of the shaft 12, the manifold 22 can be overmolded about both a part of the outer tubular member 32 and a part of the inner tubular member 30. Further due to the staggered arrangement of the inner and outer tubular members 30/32, the inner tubular member 30 extends further proximally into the body of the manifold 22 than the outer tubular member 32. The manifold 22 may include a shape, size and/or structure that is adapted for the use as desired.

For example, in the embodiment shown, the manifold 22 may include a proximal portion 48 defining a plurality of protrusions 41/43 each defining a port 42/44, respectively. The manifold 22 may also include a distal portion 50 disposed about the proximal end 16 of the shaft 12. The manifold 22 defines a plurality of lumens, for example lumens 46/47, that each extend through the manifold 22 from the shaft assembly 12 to one of the ports 42/44.

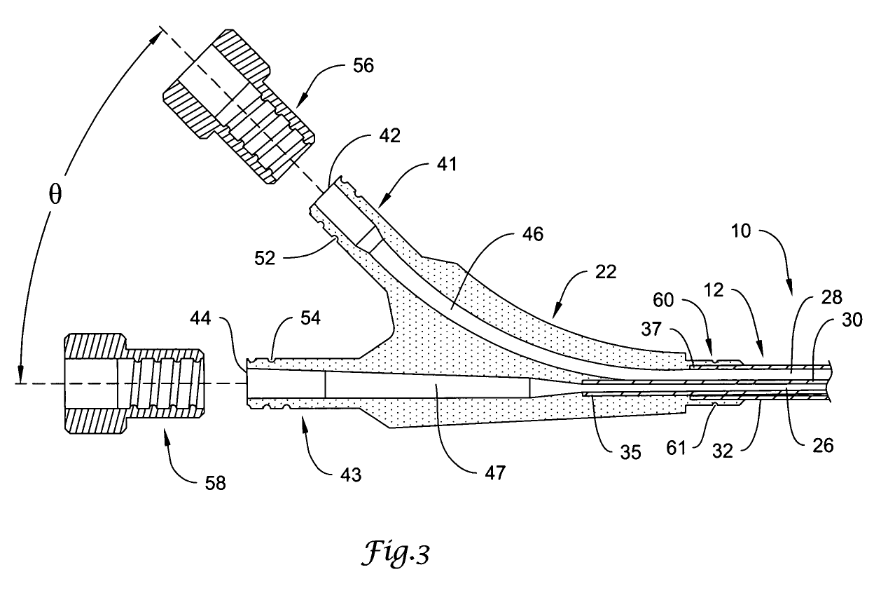

Referring now to FIG. 3, the lumen 47 extends from the proximal end 35 of the inner tubular member 30 to the port 44, thereby defining a pathway between the port 44 and the device lumen 26 through the manifold 22. The pathway may, for example, allow for a medical device, such as a guidewire or the like, to extend from the port 42 through the lumen 47 to device lumen 26. The lumen 46 extends from the proximal end 37 of the outer tubular member 32 to the port 42, thereby defining a pathway between the port 42 and the inflation lumen 28 through the manifold 22. The pathway may, for example, allow for fluid communication between the port 42 and the inflation lumen 28, such that inflation media; for example, may be delivered to the inflation lumen 28 through the port 42.

One or both of the protrusions 41/43 may include structure adapted and/or configured to allow for the connection of the manifold 22 to other structures and/or devices, such as a Luer fitting, a valve, such as a hemostatic valve, a sealing device, an inflation and/or fluid delivery device, or other fittings, valves, devices, of the like, many of which are well known in the art. The fitting, valve, device, or the like may also in turn be adapted for connection to other devices, such as a fluid delivery device and/or may be adapted to allow an additional device, such as a guidewire, to pass there through. For example, in the embodiment shown, threads 52/54 may be provided on the protrusions 41/43 for threadable connection to Luer fittings 56/58, respectively. Fitting 58 may be adapted to allow a guidewire, or other such device, to extend and/or be advanced there through in a sealing arrangement, and fitting 56 may be adapted for connection to an inflation device for delivery of inflation media to the inflation lumen 28. It should also be understood that rather than threads 28, the protrusions 41/43 may include other connecting structures, such as one or more flange, bayonet, or other connector means, or the like. In any regard, the inclusion of such connector mechanisms on the protrusions 41/43 may allow for such protrusions 41/43 to be considered connectors.

In some embodiments, the protrusions 41/43 and/or the lumens 46/47 defined by the manifold 22 may extend at an angle away from each other. For instance, the manifold 22 may include a shape such that an angle θ may be defined between the protrusions 41/43 and/or the lumens 46/47. The angle θ in some embodiments may be defined by the surfaces of the protrusions 41/43. In other embodiments, the angle θ may be defined by the axises of the lumens 46/47. For example, the lumens 46/47 defined by the protrusions 41/43 may each define a central axis, such as an input and/or output axis of each of the lumens 47/47, and the angle θ may be defined between these axises. In some embodiments, it may be desirable to provide an angle θ that is big enough such that the proximal ends of the protrusions 41/43 and/or the lumens 46/47 are separated enough to allow for easier use of the protrusions 41/43 and/or the lumens 46/47, or easier manipulation and/or attachment of other devices through or with the protrusions 41/43 and/or the lumens 46/47. For example, in some embodiments, a guidewire may be disposed within the lumen 44 during a procedure. It may be desirable to provide an angle θ that is, for example, greater than 40 degrees or so in order to allow easy manipulation of the guidewire without the protrusion 41, and/or a device (i.e. a tube connecting to a saline source) attached to the protrusion 41 getting in the way. In several embodiments, the angle θ is greater than forty degrees, while the angle θ may take on any value. In some embodiments, the angle θ is in the range of about forty to about ninety degrees, or in the range of about forty five to about fifty degrees and may be about 46.5 degrees.

Additionally, catheter 10 may also include one or more strain relief portions, for example, strain relief portion 60 disposed about the proximal portion of the shaft 12. In at least some embodiments, the strain relief portion 60 can be a portion of the unitary and/or monolithic manifold 22, meaning that the strain relief portion 60 is merely an extension of the unitary manifold 22. In other words, the unitary manifold 22 can include a strain relief portion 60 in the distal manifold portion 50 that is of unitary construction with the remainder of the manifold 22. Some embodiments, however, may include separate strain relief members, as are generally known in the art that may be attached to the shaft 12 adjacent the manifold 22. Yet other embodiments may include a separate strain relief which is disposed about the shaft 12, and thereafter, a portion of the manifold may be overmolded about a portion of the strain relief.

The strain relief portion 60 may include structure that may allow for desired flexibility characteristics, for example, to provide for a transition in flexibility between the shaft assembly 12 and the remainder of the manifold 22. For example, the strain relief portion 60 can include varying geometry, such as a tapering thickness, grooves, channels, ridges, or other such structure defined, for example, in the surface thereof to provide for the desired flexibility characteristics. In at least some embodiments, the strain relief portion 60 can be more flexible at the distal end than it is at the proximal end thereof. In the embodiment shown, the strain relief portion 60 includes one or more grooves 61 defined in the surface to provide for increased flexibility. However, it should be understood that any of a broad variety of structures may be used for the strain relief portion to achieve the desired characteristics. Some examples of strain relief configurations that may be used are disclosed in U.S. Pat. No. 6,273,404, which is incorporated herein by reference.

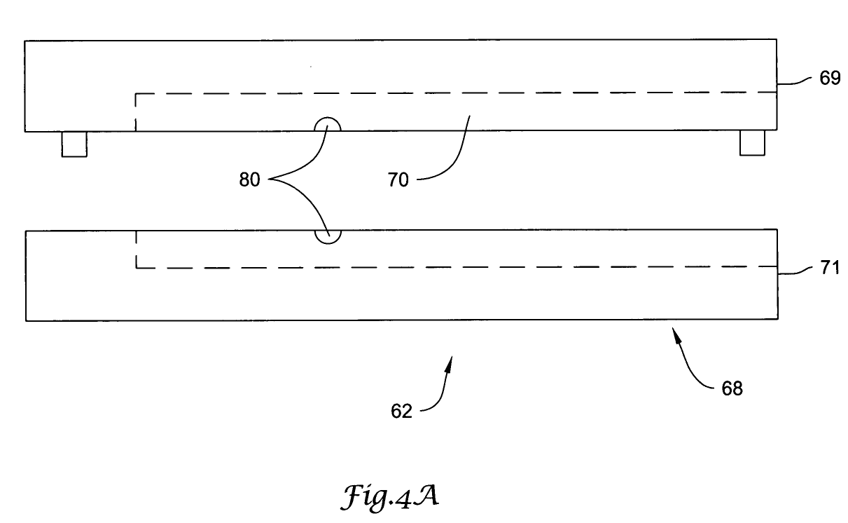

As indicated above, the manifold 22 can be overmolded and/or insert molded about the proximal portion of the shaft assembly 12. In that regard, refer now to FIG. 4A, which is a schematic side view of an example mold 62 that may be used for overmolding the manifold 22 onto the proximal end of the shaft 12. The mold 62 may be of a general type known for use in over-molding and/or insert-molding techniques. For example, the mold 62 can include a mold body 68 that may include multiple portions that when fitted together define a mold cavity 70, and can be taken apart for removal of the manifold 22 and shaft assembly 12 after the molding process is complete. For example, the mold body 68 may include two portions, such as an upper portion 69 and lower portion 71, that can be fitted together to form the mold body 68 defining the cavity 70, which is shown in phantom in FIG. 4A. The mold 62 may include an injection port 80 that can be placed in fluid communication with the mold cavity 70 such that molten molding material can be introduced into the cavity 70. The two portions 69/71 may be aligned and held together, for example using pins, clamps, bolts, or the like, as is generally known. The mold 62 may also include additional structures, such as pry bar slots, knock-out pins, or the like to aid in separating the mold portions 69/71 after molding.

FIG. 4B is a schematic top view of the lower portion 71 of the mold 62, showing a portion of the cavity 70. In some embodiments, the upper portion 69 of the mold may be generally a mirror image of the lower portion 71, and the two portions 69/71, when assembled define the mold cavity 70. In other embodiments, the upper and lower portions 69/71 may be somewhat different in size and/or shape, but when assembled, still define the desired mold cavity 70. The defined mold cavity 70 can include and/or define an area that is the general size and shape of the desired manifold 22. For example, the mold cavity 70 may allow for the production of a manifold 22 including protrusions 41/43 defining an angle θ. It should be understood, however, that any of a broad variety of manifold configurations may be defined.

FIG. 4B also shows the proximal end 16 of the shaft 12 disposed within the cavity 70, and core pins 74/76 extending from the proximal end 16 of the shaft 12 within the cavity 70. The core pins 74/76 can be used to define lumens, for example lumens 46/47, within the manifold 22 during the molding process. The core pins 74/76 may also function to substantially close off the lumens 26/28 of the shaft 12 such that molding material does not enter the lumens 26/28 during molding, thereby providing fluid communication between lumens 26/28 and lumens 46/47. For example, prior to molding, the distal end 77 of one core pin 76 can be disposed within the lumen 26 of the inner tubular member 30, while the distal end 73 of another core pin 74 can be disposed within the lumen 28 of the outer tubular member 32 between the inner and outer tubular members 30/32. The core pins 74/76 can be of appropriate size and shape to define the desired lumens 46/47 within the manifold 22 during the molding process. For example, one or both of the core pins 74/76 may be curved to provide the desired path of the lumens 74/76, and/or to achieve the desired angle, such as angle θ, between the lumens 74/76. Obviously, the core pins 74/76 must be sized and/or shaped such that they fit appropriately within the mold being used. In the embodiment shown, core pin 74 is curved, while core pin 76 is generally straight.

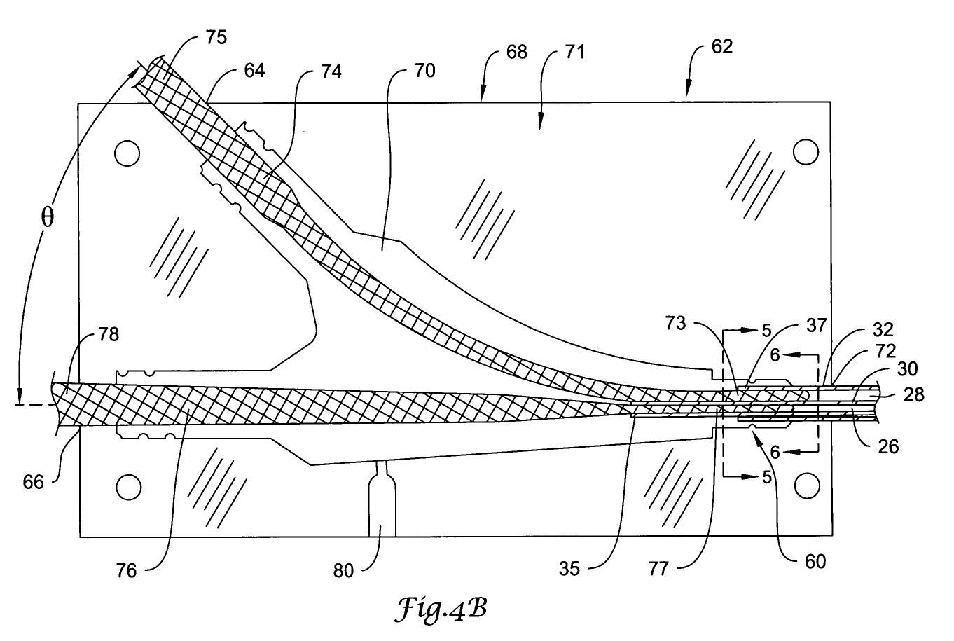

In some embodiments, one or both of the core pins 74/76 can have a cross-sectional shape that is adapted and/or configured to mate with the shape of the lumens 26/28. For example, refer now to FIG. 5, which is a cross sectional view taken along line 5-5 of FIG. 4B. As shown, the core pin 76 may include a generally circular cross-sectional shape that is adapted to mate with and substantially fill the lumen 26 defined by the inner tubular member 30. Additionally, core pin 74 may include a generally u-shaped, or half-moon shaped, cross-section that is adapted to mate with and fill a substantial portion of the lumen 28 defined by the outer tubular member 32 and the outer surface of the inner tubular member 30. This shape of the pins 74/76 can be adapted to prevent a substantial amount of molding material used in forming the manifold 22 from filling the lumens 26/28. In some embodiments, during molding, some molding material may flow a certain distance into the portion of the lumen 28 not filled by the pin 74, but will generally stop flowing after a relatively short distance as the material begins to cool. In other embodiments the core pins 74/76 may be shaped and placed so that no molding material gets within the lumens 26/28. For example, the generally circular cross-section shape of the pin 76 would substantially prevent any molding material from entering into lumen 26. The core pins 74/76 may have a continuous or varying cross sectional shapes along the lengths thereof. For example, the core pin 74 may have a generally unshaped cross sectional shape at the distal portion 73 for insertion into the lumen 28, but more proximal portions of the core pin 74 may have a generally circular, or other cross sectional shape. Additionally, the core pins 74/76 may be tapered in a continuous or stepwise fashion, for example, such that they widen and/or become larger in the proximal direction to provide for better removal of the core pins 74/76 after molding. It should be understood that the core pins 74/76 may have any of a broad variety of cross sectional shapes, sizes, lengths, dimension, or the like, dependent only upon the desired shape of the lumens being defined thereby, and the desired functioning of the core pins 74/76.

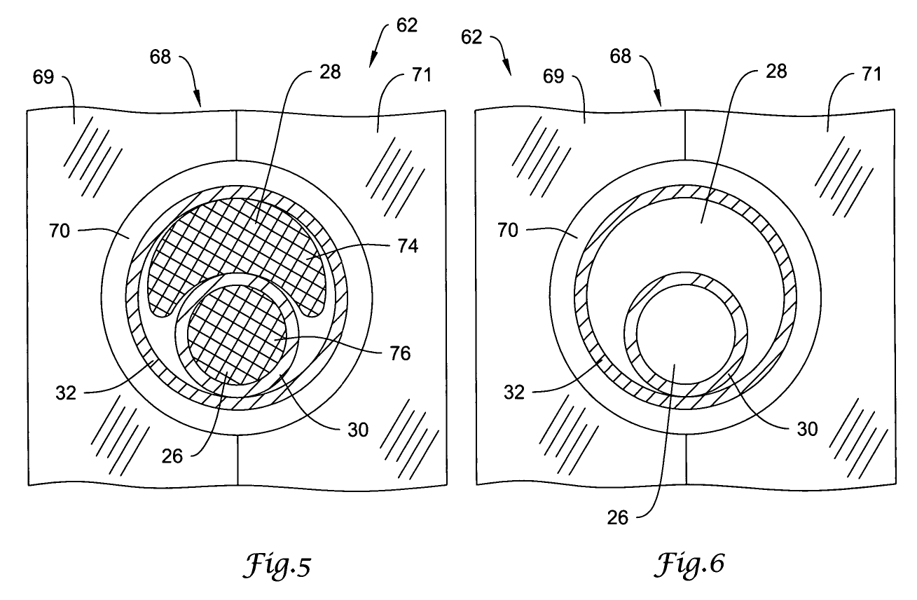

FIG. 6 is a cross sectional view taken along line 6-6 of FIG. 4B, which is at a point more distal than shown in FIG. 5. FIG. 6 shows the inner and outer tubular members 30/32 extending within the mold cavity 70, and that the lumens 26/28 remain open at this distal location.

Once the core pins 74/76 are appropriately disposed within the lumens 26/28 of the shaft 12, the entire assembly can be appropriately positioned within the mold 62, as shown in FIG. 4B. The mold 62 includes channels or openings 64, 66, and 72 defined in the body 68 of the mold 62 that are in fluid communication with the mold cavity 70. These openings 64, 66, and 72 are adapted and/or configured to mate with and maintain portions of the core pins 74/76 and the shaft 12 during the molding procedure. The proximal end 75 of the core pin 74 can be disposed in opening 64, the proximal end 78 of the core pin 76 can be disposed in opening 66, and the proximal portion 16 of the shaft 12 is disposed in opening 72. The openings 64, 66, and 72 are sized and shaped to provide sealing engagement about the core pins 74/76 and shaft 12, respectively, such that when the mold is closed, the cavity 70 is generally closed.

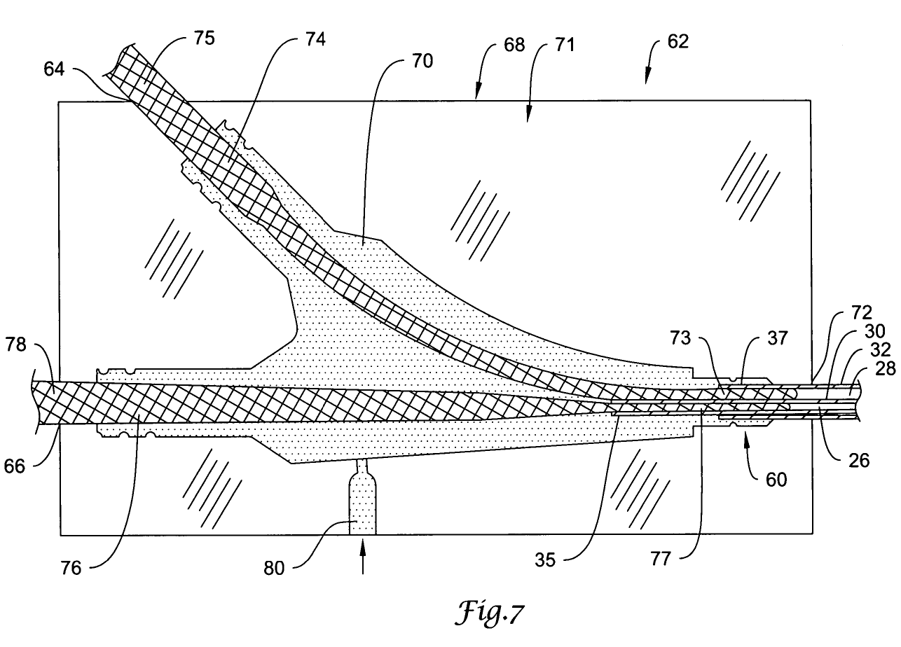

With reference now to FIG. 7, once the core pins 74/76 and the proximal portion 16 of the shaft 12 are appropriately positioned within the mold 62, a molding material, for example a molten molding material, can be introduced and/or injected into the mold cavity 70 through the port 80 to form the manifold 22. Generally, the entire mold cavity is filled with the molding material. Any of a wide variety of materials may be used, for example any of a wide variety of polymers. Some examples of polymer material may include polycarbonate, polyamide, nylon, polyether block amide (PEBA), or mixtures, combinations and/or copolymers thereof, or any other suitable material. One example commercially available suitable material is Grilamid® TR55LX produced by EMS-Chemie Holding AG/American Grilon, Inc. of Sumter, S. C. In one embodiment PEBAX 70D is chosen as the molding material.

After introduction of the molding material, the material is allowed to cure, thereby forming the manifold 22. After the manifold 22 is formed and cured sufficiently, the mold 62 may be removed and other steps in the process of fabrication performed. Again, it should be noted that the proximal end 35 of the inner tubular member 30 extends proximally from the proximal end 37 of the outer tubular member 32. As such, both the inner and outer tubular members 30/32 are attached to the manifold 22. The inner tubular member is attached by virtue of having the inner tubular member 30 extend beyond the proximal end of the outer tubular member 32.

It should be understood that this embodiment of a mold and molding technique is given by way of example only, and that a broad variety of other molds and/or molding techniques generally known may be used in overmolding and/or insert molding the manifold 22 onto the proximal end of the shaft 12.

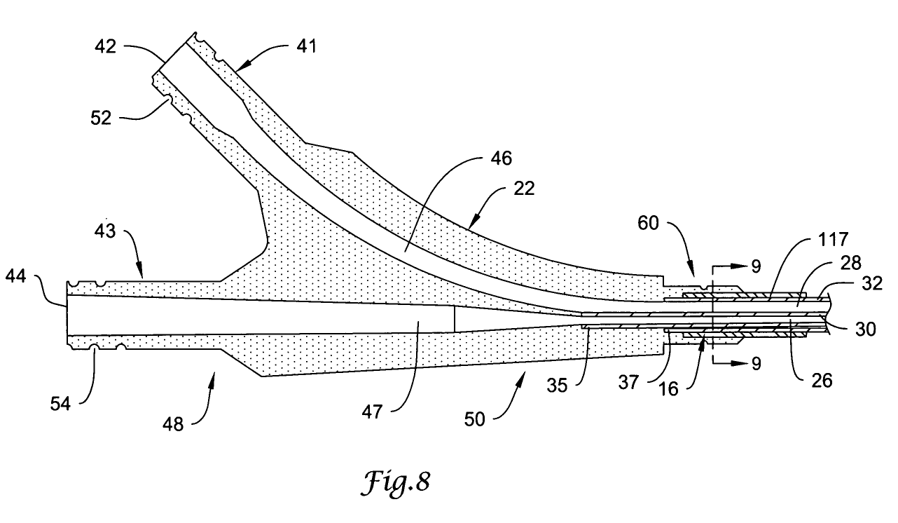

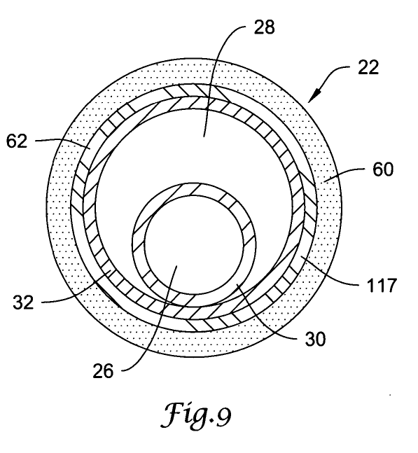

Refer now to FIG. 8, which is a section view illustrating another alternative embodiment of a manifold 22 disposed about a proximal end 16 of a catheter shaft assembly 12 similar to the embodiments described above, wherein like reference numbers indicate similar structure. This embodiment, however, includes a member and/or layer of material 117 disposed about a portion of the outer tubular member 32. A cross sectional view taken along line 9-9 of FIG. 8 is shown in FIG. 9. A portion of the member and/or layer 117 may be disposed between the outer tubular member 32 and a portion of the manifold 22, for example, a part of the strain relief portion 60 of the manifold 22.

The layer 117 may be provided over a portion of the proximal end of the outer tubular member 32 and/or the inner tubular member 30 to achieve certain desired characteristics. The embodiment shown includes a layer 117 over only the outer tubular member 32, but it should be understood that a similar layer may be disposed over the inner tubular member 30. In some embodiments, the layer 117 may provide better bonding of the manifold 22 to the tubular member 30/32. In some embodiments, the layer 117 may provide for better strength, such as burst strength, in the finished device. In some embodiments, the layer 117 may provide a protective layer to a portion of the tubular member 30/32 to provide protection and/or insulation to the tubular member 30/32 during the molding of the manifold 22 thereto. For example, when the molding material is molded onto the shaft 12, the outer tubular member 32 can in some cases be damaged (particularly if a braided steel or other support member is provided) by the heat involved in the molding process. The inclusion of a layer 117 over the proximal end of the outer tubular member 32 can improve burst strength of the finished product by protecting the outer tubular member 32 from excessive heat as well as providing additional material for containing pressure and keeping braid strands encased.

The layer 117 may be made of any of a broad variety of materials, depending upon the desired characteristics and suitable compatibility with the materials used in the other structures of the catheter. Some suitable materials include polymers, for example, the polymer materials discussed above with regard to the materials usable for the manifold 22. In some embodiments, the layer 117 may include and/or be made of the same materials as the manifold 22.

The layer 117 can be positioned and/or disposed where desired, generally over a portion of the outer tubular member 32, for example, near the proximal end of the outer tubular member 32 prior to molding of the manifold 22. Prior to molding of the manifold 22, the layer 117 may fit loosely or snugly over the outer tubular member 32. For example, in some embodiments, the layer 117 can be firmly connected to the outer tubular member 32 using adhesives, heat shrinking, or other such techniques. In other embodiments, the layer 117 may be a tubular member that is simply slipped over the proximal end of the outer tubular member 32 prior to molding. As discussed above, once these several elements are in place, the core pins can be inserted, and the entire assembly can be placed in the mold for molding of the manifold 22.

In some embodiments, the layer 1117 adheres to the outer tubular member 32 as a result of the heating that occurs during molding. In several embodiments, a cross section as shown in FIG. 9 will not include readily discernable borders between the materials making up the manifold 22, layer 1117, and/or the outside of the outer tubular member 32. The materials may re-flow during the heating that occurs during molding.

Any of the balloon catheter 10 embodiments disclosed herein may be used in a variety of different applications, for example, as an angioplasty balloon catheter, as a stent delivery catheter, or as an occlusion catheter used in conjunction with other devices in treating an aneurysm. One illustrative application is that of an angioplasty catheter. If the balloon catheter 10 is used as an angioplasty catheter, the distal end of the catheter 10 may be inserted to the femoral artery of a patient and advanced over a guidewire, such as guidewire 25, through the vasculature to a desired location near the heart. Often the process of advancing through the vasculature is aided by the use of a guide catheter which extends into the vasculature for a significant distance relative to the length of the catheter 10. The distal end of the catheter 10 is then guided over the guidewire until the balloon 24 is across a lesion or occlusion in a blood vessel. The balloon 24 is inflated to dislodge or break up the lesion. In some embodiments a stent may be placed, in a collapsed or compressed configuration, about the balloon 24 and, when the balloon 24 is inflated, the stent is expanded and placed within a blood vessel to reduce the likelihood of restenosis.

Those skilled in the art will recognize that the present invention may be manifested in a variety of forms other than the specific embodiments described and contemplated herein. Accordingly, departures in form and detail may be made without departing from the scope and spirit of the present invention as described in the appended claims.

1. A method of making a balloon catheter, the method comprising:
providing a multi-lumen catheter shaft having a proximal portion and a distal portion, the shaft including an outer tubular member defining a lumen and having a proximal end and a distal end, and an inner tubular member defining a lumen and having a proximal end and a distal end, wherein the inner tubular member is disposed within the lumen of the outer tubular member such that the proximal end of the inner tubular member extends proximally from the proximal end of the outer tubular member;
molding a manifold about the proximal portion of the multi-lumen catheter shaft such that the manifold is disposed about both the proximal end of the inner tubular member and the proximal end of the outer tubular member; and
attaching a balloon assembly to the distal portion of the shaft such that the balloon assembly is in fluid communication with at least one of the lumens.

2. The method of claim 1, wherein the proximal end of the inner tubular member extends proximally beyond the proximal end of the outer tubular member by a length of about 0.5 cm or more.

3. The method of claim 1, wherein the proximal end of the inner tubular member extends proximally beyond the proximal end of the outer tubular member by a length in the range of about 0.5 to about 3.0 cm.

4. The method of claim 1, wherein the molding step includes:
placing a first core pin into the lumen of the inner tubular member adjacent the proximal end of the inner tubular member;
placing a second core pin into the lumen of the outer tubular member between the inner and outer tubular members adjacent the proximal end of the outer tubular member;
providing a mold having a cavity including a first opening, a second opening and a third opening, the first, second and third openings being in fluid communication with the cavity;
placing the proximal end of the shaft within the mold such that the first core pin extends from the inner tubular member through the mold cavity, and passes through the first opening, the second core pin extends from the outer tubular member through the mold cavity, and passes through the second opening, and a portion of the shaft extends from within the mold cavity and passes through the third opening; and
introducing a molding material into the mold.

5. The method of claim 1, wherein the inner tubular member is disposed at least partially within the lumen of the outer tubular member such that the distal end of the inner tubular member extends distally from the distal end of the outer tubular member.

6. The method of claim 5, and wherein attaching the balloon assembly to the distal portion of the shaft includes:
providing a balloon having a proximal end and a distal end;
securing the proximal end of the balloon to the outer tubular member adjacent the distal end of the outer tubular member; and
securing the distal end of the balloon to a portion of the inner tubular member that extends distally from the distal end of the outer tubular member.

7. The method of claim 1, wherein the catheter shaft further includes a polymeric member over the proximal end of the outer tubular member, and the molding step further includes molding at least a portion of the manifold about at least a portion of the polymeric member

8. The method of claim 7 wherein, within the step of molding includes using molding material at a sufficient temperature to cause the thin polymeric member and the outer tubular member to become fused together.

9. The method of claim 1 wherein the outer tubular member includes a braided support structure.

10. The method of claim 1 wherein the balloon is adapted for use as a stent-expanding device.

11. The method of claim 1 wherein the catheter shaft further includes a polymeric member over the proximal end of the inner tubular member, and the molding step further includes molding at least a portion of the manifold about at least a portion of the polymeric member.

12. The method of claim 1 wherein the manifold includes a first protrusion including a first lumen, and a second protrusion including a second lumen.

13. The method of claim 12, wherein the first protrusion and the second protrusion form an angle of at least 40 degrees there between.

14. The method of claim 12, wherein a central axis of the first lumen and a central axis of the second lumen form an angle of at least 40 degrees there between.

15. The method of claim 1, wherein the balloon catheter is an over the wire balloon catheter.

16. A method of making a balloon catheter, the method comprising:
providing an outer tubular member having a proximal end and a distal end, and defining a lumen;
providing an inner tubular member having a proximal end and a distal end, and defining a lumen;
disposing at least a portion of the inner tubular member within the outer tubular member to define a multi-lumen catheter shaft having a proximal portion and a distal portion, wherein the proximal end of the inner tubular member extends proximally from the proximal end of the inner tubular member;
molding a manifold about the proximal portion of the multi-lumen catheter shaft such that the manifold is disposed about both the proximal end of the inner tubular member and the proximal end of the outer tubular member; and
attaching a balloon assembly to the distal portion of the shaft such that the balloon assembly is in fluid communication with at least one of the lumens.

17. A balloon catheter comprising:
a catheter shaft having a proximal portion and a distal portion, the shaft including an outer tubular member defining a lumen and having a proximal end and a distal end, and an inner tubular member defining a lumen and having a proximal end and a distal end, wherein the inner tubular member is disposed within the outer tubular member such that the proximal end of the inner tubular member extends proximally from the proximal end of the outer tubular member;
a catheter manifold molded about a portion of the outer tubular member and a portion of the inner tubular member; and
a balloon assembly attached to the distal portion of the shaft such that the balloon assembly is in fluid communication with at least one of the lumens.

18. The balloon catheter of claim 17, wherein the proximal end of the inner tubular member extends proximally beyond the proximal end of the outer tubular member by a length of about 0.5 cm or more.

19. The balloon catheter of claim 17, wherein the proximal end of the inner tubular member extends proximally beyond the proximal end of the outer tubular member by a length in the range of about 0.5 to about 3.0 cm.

20. The balloon catheter of claim 17, wherein the inner tubular member is disposed at least partially within the lumen of the outer tubular member such that the distal end of the inner tubular member extends distally from the distal end of the outer tubular member.

21. The balloon catheter of claim 20, wherein the balloon includes a proximal end secured to a portion of the outer tubular member adjacent the distal end of the outer tubular member, and the balloon includes a distal end secured to a portion of the inner tubular member that extends distally from the distal end of the outer tubular member.

22. The balloon catheter of claim 17, wherein the catheter shaft further includes a polymeric member disposed over the proximal end of the outer tubular member, wherein at least a portion of the polymeric member is disposed between the manifold and the outer tubular member.

23. The balloon catheter of claim 17, wherein the outer tubular member includes a support member.

25. The balloon catheter of claim 17, wherein the outer tubular member includes a braided support structure.

26. The balloon catheter of claim 17, wherein the balloon is adapted for use as a stent-expanding device.

27. The balloon catheter of claim 17, wherein the manifold includes a first protrusion including a first lumen, and a second protrusion including a second lumen.

28. The balloon catheter of claim 27, wherein the first protrusion and the second protrusion form an angle of at least 40 degrees there between.

29. The balloon catheter of claim 27, wherein a central axis of the first lumen and a central axis of the second lumen form an angle of at least 40 degrees there between.

30. The balloon catheter of claim 17, wherein the balloon catheter is an over the wire balloon catheter.

31. A method of making a balloon catheter, the method comprising:
providing an inner tubular member having a length, defining a lumen, and having a proximal end and a distal end;
providing an outer tubular member having a length defining a lumen and having a proximal end and a distal end;
disposing the inner tubular member within the lumen of the outer tubular member to define a shaft assembly wherein the proximal end of the inner tubular member extends further proximally than the proximal end of the outer tubular member and wherein the distal end of the inner tubular member extends further distally than the distal end of the outer tubular member;
placing a first core pin inside the proximal end of the inner tubular member;
placing a second core pin into a space defined between the inner and outer tubular members near the proximal end of the outer tubular member;
providing a mold defining a cavity having a first opening, a second opening and a third opening;
disposing at least a portion of each of the shaft assembly and core pins within the cavity of the mold such that the first core pin passes through the first opening, the second core pin passes through the second opening, and the shaft assembly passes through the third opening; and
injecting a molding material into the mold;
providing a balloon having a proximal end and a distal end;
securing the proximal end of the balloon to outer tubular member adjacent the distal end of the outer tubular member; and
securing the distal end of the balloon to the inner tubular member adjacent the distal end of the inner tubular member.
